(19) United States
(12) Patent Application Publication
(10) Pub. No.: US 2024/0261379 A1
(43) Pub. Date: Aug. 8, 2024

(54) COMPOSITION COMPRISING PEDF-DERIVED SHORT PEPTIDE, PREPARATION METHOD THEREOF, AND USE THEREOF

(71) Applicants: GRANDPHARMA (CHINA) CO., LTD., Wuhan, Hubei (CN); BRIM Biotechnology, Inc., Taipei (TW)

(72) Inventors: Lei LI, Wuhan, Hubei (CN); Yimin HU, Wuhan, Hubei (CN); Hui ZHAN, Wuhan, Hubei (CN); Caiyun XIANG, Wuhan, Hubei (CN); Lei QU, Wuhan, Hubei (CN); Wei-Cheng LIAW, San Diego, CA (US); Huei-Ling CHANG, Taipei (TW); Chih-Te CHIEN, Taipei (TW); Yi-Lun CHIANG, Taipei (TW)

(73) Assignees: GRANDPHARMA (CHINA) CO., LTD., Wuhan, Hubei (CN); BRIM Biotechnology, Inc., Taipei (TW)

(21) Appl. No.: 18/562,810

(22) PCT Filed: May 19, 2022
(86) PCT No.: PCT/CN2022/093687

§ 371 Date: Nov. 20, 2023

(30) Foreign Application Priority Data
May 19, 2021 (CN) 202110545336.6

(51) Int. Cl. A61K 38/57; A61K 47/12; A61K 47/22; A61K 47/32; A61P 27/02

(52) U.S. Cl. CPC A61K 38/57; A61K 47/12; A61K 47/22; A61K 47/32; A61P 27/02

(57) ABSTRACT

The present invention relates to a composition, a preparation method therefor, and a use thereof. The composition comprises a PEDF-derived short peptide (PDSP), a stabilizer, and a buffer agent. The pH of the composition ranges from 4 to 9. The stabilizer is a copolymer of vinyl pyrrolidone and vinyl acetate. The buffer agent is selected from a citric acid buffer system, a phosphoric acid buffer system, a boric acid buffer system, a histidine buffer system, or a combination thereof. The composition has improved stability, small irritation to eyes, and/or high bioavailability.

### TECHNICAL FIELD

The present invention relates to compositions comprising PEDF-derived short peptides (PDSP) and methods for their preparation and uses.

### BACKGROUND

Human pigment epithelium-derived factor (PEDF) is a secreted protein containing 418 amino acids with a molecular weight of approximately 50 kDa. PEDF is a multifunctional protein with multiple biological functions (see U.S. Patent Application Publication No. 2010/0047212).

Human PEDF-derived short peptides (PDSP) have been found to be promising therapeutic agents for the treatment or prevention of various diseases or conditions. For example, PDSP has been found to be effective in promoting muscle regeneration or arteriogenesis (U.S. Pat. No. 9,884,012), treating hair loss and/or hair discoloration (U.S. Pat. No. 9,938,328), treating osteoarthritis (U.S. Pat. No. 9,777,048), and preventing or improving skin aging (U.S. Pat. No. 9,815,878) or treating liver cirrhosis (U.S. Pat. No. 8,507,446).

However, formulations of these peptides were found to lack long-term stability (over months). Therefore, better compositions for this promising biopharmaceutical product are needed.

### SUMMARY OF THE INVENTION

In a first aspect, the invention provides a composition comprising PEDF-derived short peptide (PDSP), a stabilizer and a buffer; wherein the composition is in a liquid form: wherein the pH of the composition is between 4 and 9; wherein the stabilizer is a copolymer of vinylpyrrolidone and vinyl acetate: wherein the buffer is selected from a citric acid buffer system, a phosphate buffer system, a boric acid buffer system, a histidine buffer system, or a combination thereof.

In a second aspect, the present invention provides a method for preparing the above composition, which includes: a) adding a stabilizer and a buffer: b) adjusting the pH to a range of 4-9 with a base/acid; and c) adding PEDF-derived short peptide (PDSP).

In a third aspect, the present invention provides the use of the above composition in the preparation of a medicament for the treatment and/or prevention of a disease, wherein the disease is selected from the group consisting of muscle regeneration or arteriogenesis, alopecia and/or hair depigmentation, osteoarthritis, skin aging, liver cirrhosis, eye disease, or a combination thereof.

The composition comprising PEDF-derived short peptides of the present invention has improved stability (physical stability and/or chemical stability), low irritation to the eyes and/or high bioavailability.

### DETAILED DESCRIPTION

As used herein, the term “PEDF-derived short peptide (PDSP)” refers to peptides derived from PEDF and having varying numbers of amino acid residues, such as peptides having 5 to 40 amino acid residues.

Examples of PDSPs may include those shown in Table 1:

TABLE 1
Examples of PEDF-derived short peptides (PDSP)

| | | SEQ | Human PEDF |
|---|---|---|---|
| Name | Sequence | ID NO | residues |
| 39 mer | LSVATALSALSLGAEQRTESIIHRALYYDLISSPDIHGT | 1 | 82-121 |
| 34 mer | ALSALSLGAEQRTESIIHRALYYDLISSPDIHGT | 2 | 88-121 |
| 29 mer | SLGAEQRTESIIHRALYYDLISSPDIHGT | 3 | 93-121 |
| 25 mer | EQRTESIIHRALYYDLISSPDIHGT | 4 | 97-121 |
| 24 mer | SLGAEQRTESIIHRALYYDLISSP | 5 | 93-116 |
| 20 mer | SLGAEQRTESIIHRALYYDL | 6 | 93-112 |
| 18 mer | EQRTESIIHRALYYDLIS | 7 | 97-114 |
| mo29 mer | SLGAEHRTESVIHRALYYDLITNPDIHST | 8 | mouse |
| mo20 mer | SLGAEHRTESVIHRALYYDL | 9 | mouse |

Additionally, the N-terminus of PDSP can optionally be protected by acylation (e.g., acetyl or propionyl protection), and the C-terminus can optionally be protected as an amide.

The amino acid sequence identity of the peptides mentioned herein (i.e., PDSP) is at least 70%, preferably at least 80%, more preferably at least 90%. In particular, the peptides mentioned herein (i.e., PDSP) may be specifically modified to alter the characteristics of the peptide that are irrelevant to its physiological activity.

Those skilled in the art would appreciate that the peptide sequences listed in Table 1 above are for illustration only, and other sequences are also possible without departing from the scope of the present invention. Furthermore, although the above may represent some short peptides that are effective in preventing and/or treating diseases such as dry eye syndrome, shorter or longer peptides may also be used. Specifically, longer peptides may provide more favorable pharmacokinetics and/or bioavailability.

As used herein, the term “copolymer of vinylpyrrolidone and vinyl acetate” is a block polymer of varying ratios of polyvinylpyrrolidone (PVP) to polyvinyl acetate (PVAc). Usually, the copolymer of vinylpyrrolidone and vinyl acetate refers to those in which the ratio of vinylpyrrolidone building unit to vinyl acetate building unit is 10:90, 20:80, 30:70, 40:60, 50:50, 60:40, 70:30, 80:20 and/or 90:10; more specifically, examples of copolymers of vinylpyrrolidone and vinyl acetate include but are not limited to PPVVA19, PPVVA28, PPVVA37, PPVVA55, PPVVA64, PPVVA73, or a combination thereof.

As used herein, the term “citrate buffer system” refers to a buffer system comprising a substance capable of providing citrate and a base/acid (depending on the desired pH). Wherein, the substance that can provide citrate radicals can be selected from citric acid, citrate, or a combination thereof; the base is usually an inorganic base, for example, it can be selected from alkali metal hydroxide, alkali metal carbonate, alkali metal bicarbonate, or a combination thereof; and/or, the acid is usually an inorganic acid, for example, it can be selected from hydrochloric acid, phosphoric acid, or a combination thereof.

As used herein, the term “phosphate buffer system” refers to a buffer system comprising a substance capable of providing phosphate and a base/acid (depending on the desired pH). Among them, the substance that can provide phosphate can be selected from phosphoric acid, hydrogen phosphate, dihydrogen phosphate, phosphate, or a combination thereof; the base is usually an inorganic base, for example, it can be selected from alkali metal hydroxide, alkali metal carbonate, alkali metal bicarbonate, or a combination thereof; and/or, the acid is usually an inorganic acid, for example, it can be selected from hydrochloric acid, phosphoric acid, or a combination thereof.

As used herein, the term “borate buffer system” refers to a buffer system comprising a substance capable of providing borate and a base/acid (depending on the desired pH). Among them, the substance that can provide borate can be selected from boric acid, borate, or a combination thereof; the base is usually an inorganic base, for example, it can be selected from alkali metal hydroxide, alkali metal carbonate, alkali metal bicarbonate, or a combination thereof; and/or, the acid is usually an inorganic acid, for example, it can be selected from hydrochloric acid, phosphoric acid, or a combination thereof.

As used herein, the term “histidine buffer system” refers to a buffer system comprising histidine and a base/acid (the choice of whether base or acid is used depends on the desired pH), wherein the base is usually an inorganic base, for example, it can be selected from alkali metal hydroxide, alkali metal carbonate, alkali metal bicarbonate, or a combination thereof; and/or, the acid is usually an inorganic acid, for example, it can be selected from hydrochloric acid, Phosphoric acid, or a combination thereof.

As used herein, the term “alkali metal hydroxide” generally includes lithium hydroxide, sodium hydroxide, potassium hydroxide, rubidium hydroxide, cesium hydroxide, and the like: more specifically, the alkali metal hydroxide is selected from hydroxide Sodium, potassium hydroxide, or a combination thereof.

As used herein, the term “alkali metal carbonate” generally includes lithium carbonate, sodium carbonate, potassium carbonate, rubidium carbonate, cesium carbonate, and the like; more specifically, the alkali metal carbonate is selected from sodium carbonate, potassium carbonate, or a combination thereof.

As used herein, the term “alkali metal bicarbonate” generally includes lithium bicarbonate, sodium bicarbonate, potassium bicarbonate, rubidium bicarbonate, cesium bicarbonate, and the like: more specifically, the alkali metal bicarbonate is selected from bicarbonate Sodium, potassium bicarbonate, or a combination thereof.

The weight percent (w/v) stated in this application is based on the final volume of the preparation when it is in a liquid form and represents grams of each component per 100 ml of the final volume.

In a first aspect, the invention provides a composition comprising PEDF-derived short peptide (PDSP), a stabilizer and a buffer; the pH of the composition is in the range of 4-9: wherein the stabilizer is a copolymer of vinylpyrrolidone and vinyl acetate: wherein the buffer is selected from a citric acid buffer system, a phosphate buffer system, a boric acid buffer system, a histidine buffer system, or a combination thereof.

In a specific embodiment, the composition is in the form of a solution or a suspension, preferably a solution, more preferably an ophthalmic solution. Specifically, the solvent of the solution or suspension is water, preferably distilled water. Specifically, the composition is in a sterile form, preferably a sterile solution, more preferably a sterile ophthalmic solution.

In a specific embodiment, the PDSP is selected from SEQ ID NO: 1 (39-mer), SEQ ID NO: 2 (34-mer), SEQ ID NO: 3 (29-mer), SEQ ID NO: 5 (24-mer), SEQ ID NO: 6 (20-mer), SEQ ID NO: 8 (mo29-mer), SEQ ID NO: 9 (mo20-mer), or a combination thereof, wherein mo29-mer and mo20-mer are mouse PDSPs corresponding to the human 29-mer and 20-mer, respectively. Preferably, the PDSP is selected from SEQ ID NO: 3, 8, or a combination thereof.

In a specific embodiment, the stabilizer is PVPVA64.

In a specific embodiment, the citric acid buffer system consists of citric acid (or citrate) and alkali metal hydroxide/hydrochloric acid (alkali metal hydroxide or hydrochloric acid is selected depending on the desired pH). Specifically, the citric acid buffer system is citric acid (such as citric acid monohydrate) and/or sodium citrate.

In a specific embodiment, the phosphate buffer system is composed of phosphoric acid and hydrogen phosphate, hydrogen phosphate and dihydrogen phosphate, or phosphoric acid (or hydrogen phosphate or dihydrogen phosphate) and an alkali metal hydroxide. It comprises alkali metal hydroxide or hydrochloric acid depending on the desired pH. Preferably, the phosphate buffer system is selected from phosphoric acid-hydrogen phosphate, hydrogen phosphate-dihydrogen phosphate, hydrogen phosphate-hydrochloric acid, dihydrogen phosphate-alkali metal hydroxide, or a combination thereof.

In a specific embodiment, a boric acid buffer system consists of boric acid (or borate salt) and alkali metal hydroxide/hydrochloric acid (alkali metal hydroxide or hydrochloric acid is selected depending on the desired pH).

In a specific embodiment, the histidine buffer system consists of histidine and alkali metal hydroxide/hydrochloric acid (either alkali metal hydroxide or hydrochloric acid depending on the desired pH).

Preferably, in a composition of the present invention, the buffering agent is a citric acid buffering system and/or a histidine buffering system.

In a specific embodiment, the concentration of PDSP can be 0.001%-5% w/v, preferably 0.005%-1% w/v, more preferably 0.01%-0.05% w/v, most preferably 0.02%-0.04% w/v, for example 0.03% w/v.

In another embodiment, the concentration of PDSP is generally 0.01-50 mg/ml, preferably 0.05-10 mg/ml, more preferably 0.1-0.5 mg/ml, more preferably 0.2-0.4 mg/ml, such as 0.3 mg/ml.

In a specific embodiment, the concentration of the stabilizer may be 0.1%-4% w/v, preferably 0.1%-3% w/v, more preferably 0.3%-2% w/v, most preferably 0.5%-1.5% w/v.

In another embodiment, the concentration of the stabilizer may be 1-40 mg/ml, preferably 1-30 mg/ml, more preferably 3-20 mg/ml, most preferably 5-15 mg/ml.

In a specific embodiment, the concentration of the buffer may be between 1 mM and 200 mM, preferably between 5 and 150 mM, more preferably between 10 and 100 mM, and most preferably between 40 and 60 mM.

In another embodiment, the concentration of buffer may be 0.005%-5% w/v, preferably 0.03%-3.5% w/v, more preferably 0.06%-2.5% w/v, most preferably 0.2% -1.5% w/v.

In yet another embodiment, the concentration of the buffer may be 0.05-50 mg/ml, preferably 0.3-35 mg/ml, more preferably 0.6-25 mg/ml, most preferably 2-15 mg/ml.

In a specific embodiment, the pH of the composition is preferably in the range of 6-8, more preferably in the range of 6.5-7.5, most preferably in the range of 7-7.5.

In a specific embodiment, the composition also contains other excipient additives. Specifically, the other excipient additives may be selected from osmolarity regulators, bacteriostatic agents, suspending agents, or a combination thereof, and are preferably selected from osmolarity regulators, bacteriostatic agents, or a combination thereof.

Specifically, the osmolarity regulator may be selected from sorbitol, sodium chloride, potassium chloride, glucose, boric acid, or a combination thereof, and is preferably selected from sorbitol, glucose, or a combination thereof. Specifically, the concentration of the osmolarity regulator is usually 0.005%-5% w/v, preferably 0.1%-4% w/v, more preferably 1%-3.5% w/v, and most preferably 2%-3% w/v. Alternatively, the concentration of the osmolarity regulator is usually 0.05-50 mg/ml, preferably 1-40 mg/ml, more preferably 10-35 mg/ml, most preferably 20-30 mg/ml: Alternatively, the concentration of the osmolarity regulator may be ImM-500 mM, preferably 10-400 mM, more preferably 50-300 mM, most preferably 100 mM-200 mM.

Specifically, the bacteriostatic agent can be selected from quaternary ammonium salts, organic mercury, amidines, alcohols, esters, or a combination thereof, and is preferably selected from benzalkonium chloride, benzalkonium bromide, thimerosal, nitric acid mercury, chlorhexidine, benzyl alcohol, chlorobutanol, ethylparaben, methylparaben, propylparaben, butylparaben, or a combination thereof, more preferably selected from benzalkonium chloride, benzalkonium bromide, thimerosal, chlorhexidine, chlorobutanol, ethyl hydroxyphenyl, or a combination thereof, most preferably selected from benzalkonium chloride, benzalkonium bromide, thimerosal, or a combination thereof. Specifically, the concentration of the bacteriostatic agent is usually 0.004%-0.025% w/v, preferably 0.008%-0.021% w/v, more preferably 0.01%-0.02% w/v: or wherein the concentration of the bacterial agent is usually 0.004-0.25 mg/ml, preferably 0.08-0.21 mg/ml, and more preferably 0.1-0.2 mg/ml.

Specifically, the suspending agent may be selected from small-molecule suspending agents, large-molecule suspending agents, silicates, thixotropes, or a combination thereof. Specifically, the small-molecule suspending agent can be selected from glycerol, syrup, or a combination thereof: the large-molecule suspending agent can be selected from gums (such as acacia gum, tragacanth gum, peach gum, or a combination thereof), plant mucilage and polysaccharides (such as sodium alginate, agar, starch, pectin, carrageenan, chitosan, or a combination thereof), cellulose derivatives (such as methylcellulose or its salts, carboxymethyl fiber cellulose or its salt, hydroxypropyl cellulose or its salt, hydroxyethyl cellulose or its salt, or a combination thereof), or a combination thereof: the silicate can be selected from bentonite, magnesium aluminum silicate, aluminum silicate, or a combination thereof; and/or the thixotrope can be selected from citrate, hydrogen citrate, tartrate, hydrogen tartrate, phosphate, AlCl₃, or a combination thereof. Specifically, the concentration of the suspending agent is usually 0.1%-15% w/v, preferably 0.1-10% w/v, more preferably 1-5% w/v; or the concentration of the suspending agent It is usually 1-150 mg/ml, preferably 1-100 mg/ml, and more preferably 10-50 mg/ml.

In a specific embodiment of the first aspect of the invention, there is provided a composition comprising a PEDF-derived short peptide (PDSP), a stabilizer and a buffer: the pH of the composition is in the range of 6.5-7.5; wherein the stabilizer is a copolymer of vinylpyrrolidone and vinyl acetate; the concentration of the stabilizer is 3-20 mg/ml: wherein the buffer is selected from a citric acid buffer system or a histidine buffer system: the composition further includes an osmolarity regulator, wherein the concentration of the osmolarity regulator is 100 mM-200 mM.

In another specific embodiment of the first aspect of the invention, there is provided a composition comprising a PEDF-derived short peptide (PDSP), a stabilizer and a buffer: the pH of the composition is in the range of 6.5-7.5; wherein the stabilizer is a copolymer of vinylpyrrolidone and vinyl acetate: the concentration of the stabilizer is 3-20 mg/ml; wherein the buffer is selected from a citric acid buffer system or a histidine buffer system: the composition further includes an osmolarity regulator, wherein the concentration of the osmolarity regulator is 100 mM-200 mM: the composition is in the form of a solution, more preferably an ophthalmic solution.

In yet another specific embodiment of the first aspect of the invention, there is provided a composition comprising a PEDF-derived short peptide (PDSP), a stabilizer and a buffer; the pH of the composition is in the range of 6.5-7.5: the stabilizer is PPVVA64; the concentration of the stabilizer is 3-20 mg/ml: wherein the buffer is selected from a citric acid buffer system or a histidine buffer system; the concentration of the buffer is 40 mM-60 mM; the composition also includes an osmolarity regulator, wherein the concentration of the osmolarity regulator is 100 mM-200 mM.

In yet another specific embodiment according to the first aspect of the invention, there is provided a composition comprising a PEDF-derived short peptide (PDSP), a stabilizer and a buffer: the pH of the composition is in the range of 6.5-7.5; the stabilizer is PPVVA64: the concentration of the stabilizer is 3-20 mg/ml; wherein the buffer is selected from a citric acid buffer system or a histidine buffer system; the concentration of the buffer is 40 mM-60 mM: the composition further includes an osmolarity regulator, wherein the concentration of the osmolarity regulator is 100 mM-200 mM: the composition is in the form of a solution, more preferably an ophthalmic solution.

In yet another specific embodiment of the first aspect of the invention, there is provided a composition comprising a PEDF-derived short peptide (PDSP), a stabilizer and a buffer; the pH of the composition is in the range of 6.5-7.5: the stabilizer is PPVVA64: the concentration of the stabilizer is 3-20 mg/ml; wherein the buffer is selected from a citric acid buffer system or a histidine buffer system; the concentration of the buffer is 40 mM-60 mM: the composition further includes an osmolarity regulator, wherein the osmolarity regulator is sorbitol, and the concentration of the osmolarity regulator is 100 mM-200 mM: the PDSP is selected from SEQ ID NO: 3, 8, or a combination thereof; the composition is in the form of a solution, more preferably an ophthalmic solution.

In the second aspect, the invention provides a method for preparing the above composition, which includes: a) adding a stabilizer and a buffer: b) adjusting the pH to a range of 4-9; and c) adding PEDF-derived short peptide (PDSP).

In a method of the invention, the pH of the composition and the components such as stabilizers, buffers, PEDF-derived short peptides (PDSP) and/or their concentrations are as described above.

In a specific embodiment, a method includes: a) adding a stabilizer and a buffer, adding water and stirring or sonicate until they dissolved: b) adjusting the pH to a range of 4-9 with alkali/acid; and c) adding PEDF-derivatized short peptide (PDSP), stir or sonicate until it dissolved. Preferably, the dissolution in steps a) and/or c) is achieved by stirring: more preferably, the dissolution in steps a) and/or c) is achieved by manual stirring or stirring in a magnetic stirrer by adding a magnet. Preferably, the base in step b) is an inorganic base, for example, it can be selected from alkali metal hydroxides, alkali metal carbonates, alkali metal bicarbonates, or a combination thereof. Preferably, the acid in step b) is an inorganic acid or an organic acid, for example, it can be selected from hydrochloric acid, phosphoric acid, citric acid, or a combination thereof.

In another specific embodiment, the method further comprises d) filtering through a 0.22 μm filter. Preferably, step d) is performed at room temperature. Specifically, the filter may be a syringe filter. Specifically, steps a), b) and/or c) may be performed at room temperature.

In a third aspect, the present invention provides a use of the above composition in the preparation of a medicament for the treatment and/or prevention of a disease, wherein the disease is selected from the group consisting of muscle regeneration or arteriogenesis, alopecia and/or hair depigmentation, osteoarthritis, skin aging, liver cirrhosis, eye disease, or a combination thereof. Preferably, the eye disease is selected from dry eye disease and symptoms related to dry eye disease (such as ocular surface damage and ocular surface inflammation caused by dry eye disease).

The various embodiments or solutions at different preference levels described herein can be combined arbitrarily, unless otherwise stated.

The present invention will be illustrated below in the form of specific examples. However, these examples should not be understood as limiting the scope of the subject matter of the present invention. All technologies implemented based on the above contents of the present invention belong to the scope of the present invention. The compounds or reagents used in the following examples can be purchased through commercial channels or prepared by conventional methods known to those skilled in the art, and the experimental instruments used can be purchased through commercial channels.

### EXAMPLE

### Example 1

At room temperature, weigh 0.80 g PVPVA64 (trade name: Kollidon® VA 64, purchased from BASF, Germany) and 0.31 g histidine (purchased from Wuxi Bikang Bioengineering Co., Ltd.), add 30 ml of water and stir until dissolved; use 2N NaOH solution or IN HCl to adjust the pH to pH7.5: add 12 mg PDSP (such as 29-mer, purchased from BRIM Biotechnology, Inc.), stir until it dissolved, add water to adjust the volume to 40 ml: filter through a 0.22 μm syringe filter (purchased from Sango Biotech (Shanghai) Co., Ltd.) to obtain the Formulation/preparation 1 of the present invention.

### Example 2

At room temperature, weigh 0.60 g PVPVA64 (trade name: Kollidon®) VA 64, purchased from BASF, Germany) and 0.31 g histidine (purchased from Wuxi Bikang Bioengineering Co., Ltd.), add 30 ml of water and stir until dissolved; use 2N NaOH solution or IN HCl to adjust the pH to pH7.5: add 12 mg PDSP (such as 29-mer, purchased from BRIM Biotechnology. Inc.), stir until it dissolved, add water to adjust the volume to 40 ml; filter through a 0.22 μm syringe filter (purchased from Sango Biotech (Shanghai) Co., Ltd.) to obtain Formulation 2 of the present invention.

### Example 3

At room temperature, weigh 0.40 g PVPVA64 (trade name: Kollidon& VA 64, purchased from BASF, Germany) and 0.31 g histidine (purchased from Wuxi Bikang Bioengineering Co., Ltd.), add 30 ml of water and stir until dissolved; use 2N NaOH solution or IN HCl to adjust the pH to pH7.5; add 12 mg PDSP (such as 29-mer, purchased from BRIM Biotechnology, Inc.), stir until it dissolved, add water to adjust the volume to 40 ml; filter through a 0.22 μm syringe filter (purchased from Sango Biotech (Shanghai) Co., Ltd.) to obtain Formulation 3 of the present invention.

### Example 4

At room temperature, weigh 0.12 g PVPVA64 (trade name: Kollidon R) VA 64, purchased from BASF. Germany) and 0.31 g histidine (purchased from Wuxi Bikang Bioengineering Co., Ltd.), add 30 ml of water and stir until dissolved: use 2N NaOH solution or IN HCl to adjust the pH to pH7.5: add 12 mg PDSP (such as 29-mer), stir until it dissolved, add water to make the volume to 40 ml; pass through a 0.22 μm syringe filter (purchased from Sango Biotech (Shanghai) Co., Ltd.) to obtain Formulation 4 of the present invention.

### Example 5

At room temperature, weigh 40 mg PVPVA64 (trade name: KollidonR VA 64, purchased from BASF, Germany) and 0.31 g histidine (purchased from Wuxi Bikang Bioengineering Co., Ltd.), add 30 ml of water and stir until dissolved; use 2N NaOH solution or IN HCl to adjust the pH to pH7.5: add 12 mg PDSP (such as 29-mer, purchased from BRIM Biotechnology, Inc.), stir until it dissolved, add water to adjust the volume to 40 ml; filter through 0.22 μm syringe filter (purchased from Sango Biotech (Shanghai) Co., Ltd.) to obtain Formulation 5 of the present invention.

### Example 6

At room temperature, weigh 0.80 g PVPVA64 (trade name: Kollidon®) VA 64, purchased from BASF, Germany) and 0.31 g histidine (purchased from Wuxi Bikang Bioengineering Co., Ltd.), add 30 ml of water and stir until dissolved; use 2N NaOH solution or IN HCl to adjust the pH to pH7.0; add 12 mg PDSP (such as 29-mer, purchased from BRIM Biotechnology, Inc.), stir until it dissolved, add water to adjust the volume to 40 ml; filter through a 0.22 μm syringe filter (purchased from Sango Biotech (Shanghai) Co., Ltd.) to obtain Formulation 6 of the present invention.

### Example 7

At room temperature, weigh 0.80 g PVPVA64 (trade name: Kollidon® VA 64, purchased from BASF, Germany) and 0.31 g histidine (purchased from Wuxi Bikang Bioengineering Co., Ltd.), add 30 ml of water and stir until dissolved: use 2N NaOH solution or IN HCl to adjust the pH to pH7.5: add 4 mg PDSP (such as 29-mer, purchased from BRIM Biotechnology, Inc.), stir until it dissolved, add water to adjust the volume to 40 ml; filter through a 0.22 μm syringe filter (purchased from Sango Biotech (Shanghai) Co., Ltd.) to obtain Formulation 7 of the present invention.

### Example 8

At room temperature, weigh 0.80 g PVPVA64 (trade name: Kollidon® VA 64, purchased from BASF, Germany) and 0.31 g histidine (purchased from Wuxi Bikang Bioengineering Co., Ltd.), add 30 ml of water and stir until dissolved; use 2N NaOH solution or IN HCl to adjust the pH to pH7.5: add 20 mg PDSP (such as 29-mer, purchased from BRIM Biotechnology, Inc.), stir until it dissolved, add water to adjust the volume to 40 ml; filter through a 0.22 μm syringe filter (purchased from Sango Biotech (Shanghai) Co., Ltd.) to obtain Formulation 8 of the present invention.

### Example 9

At room temperature, weigh 0.80 g PVPVA64 (trade name: Kollidon®) VA 64, purchased from BASF, Germany) and 0.42 g citric acid monohydrate (purchased from Shanghai Aladdin Biochemical Technology Co., Ltd.), add 30 ml of water and stir until dissolved: add 2N NaOH solution or IN hydrochloric acid to adjust the pH to pH7.5: add 12 mg PDSP (such as 29-mer, purchased from BRIM Biotechnology. Inc.), stir until it dissolved, add water to make the volume to 40 ml: filter through a 0.22 μm syringe filter ((purchased from Sango Biotech (Shanghai) Co., Ltd.) to obtain Formulation 9 of the present invention.

### Example 10

At room temperature, weigh 0.60 g PVPVA64 (trade name: Kollidon®) VA 64, purchased from BASF, Germany) and 0.43 g sodium citrate (purchased from Bailingwei Technology Co., Ltd.), add 30 ml of water and stir until dissolved: use 2N NaOH or IN hydrochloric acid to adjust the pH to pH7.5: add 12 mg PDSP (such as 29-mer, purchased from BRIM Biotechnology. Inc.), stir until it dissolved, add water to adjust the volume to 40 ml: filter through 0.22 μm syringe filter (purchased from Sango Biotech (Shanghai) Co., Ltd.) to obtain the Formulation 10 of the present invention.

### Example 11

At room temperature, weigh 0.40 g PVPVA64 (trade name: Kollidon& VA 64, purchased from BASF, Germany) and 0.42 g citric acid monohydrate (purchased from Shanghai Aladdin Biochemical Technology Co., Ltd.), add 30 ml of water and stir until dissolved: add 2N NaOH solution or IN hydrochloric acid to adjust the pH to pH7.5: add 12 mg PDSP (such as 29-mer, purchased from BRIM Biotechnology, Inc.), stir until dissolved, add water to make the volume to 40 ml: filter through a 0.22 μm syringe filter (purchased from Sango Biotech (Shanghai) Co., Ltd.) and filtered to obtain Formulation 11 of the present invention.

### Example 12

At room temperature, weigh 0.12 g PVPVA64 (trade name: Kollidon®) VA 64, purchased from BASF, Germany) and 0.42 g citric acid monohydrate (purchased from Shanghai Aladdin Biochemical Technology Co., Ltd.), add 30 ml of water and stir until dissolved: add 2N NaOH solution or IN hydrochloric acid to adjust the pH to pH7.5: add 12 mg PDSP (such as 29-mer), stir until dissolved, add water to make the volume to 40 ml: filter through a 0.22 μm syringe filter (purchased from Sango Biotech (Shanghai) Co., Ltd. Company) to obtain the Formulation 12 of the present invention.

### Example 13

At room temperature, weigh 40 mg PVPVA64 (trade name: Kollidon& VA 64, purchased from BASF, Germany) and 0.42 g citric acid monohydrate (purchased from Shanghai Aladdin Biochemical Technology Co., Ltd.), add 30 ml of water and stir until dissolved: use 2N NaOH solution or IN HCl to adjust the pH to pH7.5: add 12 mg PDSP (such as 29-mer, purchased from BRIM Biotechnology, Inc.), stir until it dissolved, add water to adjust the volume to 40 ml: filter through 0.22 μm syringe filter (purchased from Sango Biotech (Shanghai) Co., Ltd.), to obtain Formulation 13 of the present invention.

### Example 14

At room temperature, weigh 0.80 g PVPVA64 (trade name: Kollidon® VA 64, purchased from BASF. Germany), 0.42 g citric acid monohydrate (purchased from Shanghai Aladdin Biochemical Technology Co., Ltd.) and 1.00 g sorbitol (purchased from Beijing Bailingwei Technology Co., Ltd.), add 30 ml of water and stir until dissolved; use 2N NaOH solution or IN hydrochloric acid to adjust the pH to pH7.5: add 12 mg PDSP (such as 29-mer, purchased from BRIM Biotechnology, Inc.), stir until it dissolved, add water to adjust the volume to 40 ml: filter through a 0.22 μm syringe filter (purchased from Sango Biotech (Shanghai) Co., Ltd.) to obtain Formulation/preparation 14 of the present invention.

### Example 15

At room temperature, weigh 0.80 g PVPVA64 (trade name: Kollidon®) VA 64, purchased from BASF, Germany) and 0.43 g citric acid monohydrate (purchased from Shanghai Aladdin Biochemical Technology Co., Ltd.), add 30 ml of water and stir until dissolved: add 2N NaOH solution or IN hydrochloric acid to adjust the pH to pH7.0; add 12 mg PDSP (such as 29-mer, purchased from BRIM Biotechnology, Inc.), stir until it dissolved, add water to make the volume to 40 ml; filter through a 0.22 μm syringe filter ((purchased from Sango Biotech (Shanghai) Co., Ltd.) and filtered to obtain Formulation/preparation 15 of the present invention.

### Comparative Example 1

At room temperature, weigh 0.31 g histidine (purchased from Wuxi Bikang Bioengineering Co., Ltd.), add 30 ml of water and stir until dissolved: adjust the pH to pH7.0 with 2N NaOH solution or IN hydrochloric acid: add 12 mg PDSP (such as 29-mer, purchased from BRIM Biotechnology, Inc.), stir until it dissolved, add water to adjust the volume to 40 ml; filter through a 0.22 μm syringe filter (purchased from Sango Biotech (Shanghai) Co., Ltd.) to obtain comparative Formulation/preparation 1.

### Comparative Example 2

At room temperature, weigh 0.12 g histidine (purchased from Wuxi Bikang Bioengineering Co., Ltd.) and 1.71 g nicotinamide (purchased from Xi′ an Yuelai Pharmaceutical Technology Co., Ltd.), add 30 ml of water and stir until dissolved; use 2N NaOH solution or IN HCl to adjust the pH to pH7.0: add 12 mg PDSP (such as 29-mer, purchased from BRIM Biotechnology, Inc.), stir until it dissolved, add water to adjust the volume to 40 ml: filter through 0.22 μm syringe filter (purchased from Sango Biotech (Shanghai) Co., Ltd.) to obtain comparative Formulation/preparation 2.

### Comparative Example 3

At room temperature, weigh 0.10 g boric acid (purchased from Shaanxi Panlong Pharmaceutical Logistics Co., Ltd.) and 1.12 g glycerin (purchased from Beijing Bailingwei Technology Co., Ltd.), add 30 ml of water and stir until dissolved: use 2N NaOH solution or IN hydrochloric acid to adjust the pH to pH7.0; add 12 mg PDSP (such as 29-mer, purchased from BRIM Biotechnology, Inc.), stir until it dissolved, add water to adjust the volume to 40 ml: filter through 0.22 μm syringe filter (purchased from Sango Biotech (Shanghai) Co., Ltd.) to obtain comparative Formulation/preparation 3.

### Comparative Example 4

At room temperature, weigh 0.10 g boric acid (purchased from Shaanxi Panlong Pharmaceutical Logistics Co., Ltd.) and 2.38 g sorbitol (purchased from Xi′ an Taihua Pharmaceutical Technology Co., Ltd.), add 30 ml of water and stir until dissolved: use 2N NaOH solution or IN hydrochloric acid to adjust the pH to pH7.0; add 12 mg PDSP (such as 29-mer, purchased from BRIM Biotechnology, Inc.), stir until it dissolved, add water to adjust the volume to 40 ml: filter through 0.22 μm syringe filter (purchased from Sango Biotech (Shanghai) Co., Ltd.) to obtain comparative Formulation/preparation 4.

### Comparative Example 5

At room temperature, weigh 0.12 g histidine (purchased from Wuxi Bikang Bioengineering Co., Ltd.), 0.73 g nicotinamide (purchased from Xi′ an Yuelai Pharmaceutical Technology Co., Ltd.) and 1.02 g sorbitol (purchased from Xi′ an Taihua Pharmaceutical Technology Co., Ltd.), add 30 ml of water and stir until dissolved: use 2N NaOH solution or IN hydrochloric acid to adjust the pH to pH7.0; add 12 mg of PDSP (such as 29-mer, purchased from BRIM Biotechnology, Inc.), stir until it dissolved, and add water to 40 ml; filter through a 0.22 μm syringe filter (purchased from Sango Biotech (Shanghai) Co., Ltd.) to obtain comparative Formulation/preparation 5.

### Example 16: Forced aggregation experiment

Place the same volume (such as 40 ml) of Comparative Formulation/Preparation 2 and the Formulation/preparations prepared in Examples 1-6 and 9-15 of the present application into a flask, add a 1.5 cm magnet, and stir in a magnetic stirrer at room temperature at 1150 rpm (revolutions per Minute, rpm), observe with the naked eye any visible foreign matter appears in the solution, and using a particle detector (GWJ-16 type particle detector purchased from Tianda Tianfa Technology Co., Ltd.) at different time points (0 hours, 24 hours, 48 hours, or 72 hours) to measure insoluble particles.

The inventor focused on investigating the effects of the concentrations of the stabilizer (PVPVA64), the osmolarity regulator, and the pH in the formulations/preparations of the present invention in the forced aggregation experiments. Specifically, the order of turbidity observed with the naked eye at 72 hours between Comparative Formulation/Preparation 2 and Formulation/Preparations 1-6 of the present invention or Formulation/Preparations 9-15 of the present invention is as follows:

Formulation 5 of the present invention>Formulation 2 of the present invention>Formulation 4 of the present invention>Formulation 3 of the present invention>Formulation 2 of the present invention>Formulation 1 of the present invention Formulation 6 of the present invention:

Formulation 13 of the present invention>Comparative Formulation 2>Formulation 12 of the present invention>Formulation 11 of the present invention>Formulation 10 of the present invention>Formulation 9 of the present invention>Formulation 14 of the present invention≈Formulation 15 of the present invention.

In addition, since the aggregation phenomenon was observed with the naked eye in the tested solution after 72 hours. However, because the first measurement result of insoluble particles was not obvious, a 1 μm range was added to the second test. It was found that most of the aggregation was concentrated in the 1 μm range.

For example, the remeasurement results of insoluble particles at 72 hours for inventive formulations 1˜4 and 14-15 and comparative formulation 2 are as follows:

| | Particle | | | | | 72 hr. |
|---|---|---|---|---|---|---|
| Formulation | size | 0 hr. | 24 hr. | 48 hr. | 72 hr. | (repeat) |
| Comparative | ≥1 μm | — | — | — | — | 4378.0 |
| Formulation 2 | ≥10 μm | 2 | 6.66 | 8.66 | 10.66 | 13.33 |
| | ≥25 μm | 0 | 0 | 0 | 0 | 2.66 |
| Invention | ≥1 μm | — | — | — | — | 795.33 |
| Formulation 1 | ≥10 μm | 3.33 | 4.66 | 4 | 6.66 | 11.33 |
| | ≥25 μm | 0 | 0 | 0 | 0.66 | 1.33 |
| Invention | ≥1 μm | — | — | — | — | 688.0 |
| Formulation 2 | ≥10 μm | 4 | 6 | 10 | 10.66 | 1.33 |
| | ≥25 μm | 1.33 | 0.66 | 0.66 | 0.66 | 0.66 |
| Invention | ≥1 μm | — | — | — | — | 1526.0 |
| Formulation 3 | ≥10 μm | 0.66 | 4.66 | 9.33 | 6.66 | 10.0 |
| | ≥25 μm | 0 | 0 | 1.33 | 0 | 0.66 |
| Invention | ≥1 μm | — | — | — | — | 2196.66 |
| Formulation 4 | ≥10 μm | 2.66 | 12.66 | 11.33 | 26.66 | 36.66 |
| | ≥25 μm | 0 | 0 | 0.66 | 0 | 2.0 |
| Invention | ≥1 μm | — | — | — | — | 1262.0 |
| Formulation 14 | ≥10 μm | 0 | 5.33 | 5.33 | 5.33 | 15.33 |
| | ≥25 μm | 0 | 0 | 0.66 | 0.66 | 0 |
| Invention | ≥1 μm | — | — | — | — | 1269.33 |
| Formulation 15 | ≥10 μm | 0.66 | 6.0 | 8.0 | 17.33 | 26.0 |
| | ≥25 μm | 0 | 1.33 | 1.33 | 0.66 | 1.33 |

### Example 17 ASAP (Accelerated Stability Assessment Program) Experiment

Put the same volume (such as 40 ml) of comparative formulation 1-5 and the formulation prepared in Example 6 of the present invention into a stability test box (model ZSW-100˜ZSW-2000, purchased from Zhenxiang Mechanical and Electrical Technology (Shanghai) Co., Ltd.) to accelerate degradation at different temperatures for different times (1 week and 2 weeks at 40° ° C., 3 days and 6 days at)60° ° C. Then, at 0 days, the first acceleration time point (such as 1 week at)40° ° C., and the second acceleration time point (such as 2 weeks of acceleration at 40° C., 6 days of acceleration at 60° C.), the purities of the corresponding PDSP in the tested preparations were assessed by HPLC. The purity difference at the first acceleration time point (i.e., the purity at day 0 minus the purity at the first acceleration time point) and the purity difference at the second acceleration time point (i.e., the purity at day 0 minus the purity at the second acceleration time point) were used to evaluate the chemical stability of the preparations.

The specific detection conditions and steps of the HPLC method (according to the Chinese Pharmacopoeia 2015 Edition, Part Four, General Chapter <0512>>) are as follows:

Use superficially porous medium as the filling agent (Agilent Advance Bio Peptide Map 4.6 mm×150 mm, 2.7 μm, or a chromatographic column with equivalent performance); use 0.1% trifluoroacetic acid as the mobile phase A, and [(methanol/acetonitrile/water=5:4:1)+0.085% trifluoroacetic acid] as the mobile phase B, and perform gradient elution according to the following table. The flow rate is 1.0 ml per minute: the column temperature is 50° C.; and the detection wavelength is 220 nm.

| Time (min) | Mobile phase A % | Mobile phase B % |
|---|---|---|
| 0 | 65 | 35 |
| 15 | 30 | 70 |
| 16 | 65 | 35 |
| 25 | 65 | 35 |

Precisely measure 10 μl of the preparation after completing the accelerated degradation experiment, inject it into the liquid chromatograph, and record the chromatogram. Calculate the peak areas according to the external standard method to obtain the purities of PDSP in the preparations.

The specific results are as follows:

| | Purity changes after 60° C. | Purity changes after 60° C. |
|---|---|---|
| Preparation | acceleration for 3 days | acceleration for 6 days |
| Comparative preparation 1 | 6.70 | 15.66 |
| Comparative preparation 2 | 7.26 | 12.90 |
| Comparative preparation 3 | 8.03 | 15.27 |
| Comparative preparation 4 | 12.96 | 17.15 |
| Comparative preparation 5 | 7.27 | 15.73 |
| Preparation 6 of the invention | 6.47 | 11.16 |

The above results show that formulation 6 of the invention has improved chemical stability, as compared to comparative formulations 1, 2, 3, 4 or 5.

| | Purity changes after 40° C. | Purity changes after 40° C. |
|---|---|---|
| Formulation | acceleration for 1 week | acceleration for 2 weeks |
| Comparative Formulation 2 | 1.64 | 2.68 |
| Comparative Formulation 5 | 1.41 | 2.69 |
| Formulation 15 of the invention | 1.36 | 2.57 |

The above results show that compared with comparative formulation 2 or 5, formulation 15 of the present invention has equivalent or better chemical stability.

### Example 18

Materials:

Sodium carboxymethylcellulose (CMC) and periodic acid Schiff (PAS) reagent were both from Sigma-Aldrich (St. Louis, MO, USA). Balanced salt solution (BSS; Alcone) containing CMC (1% w/v) was used as vehicle.

### Animals:

7-8 weeks old female C57BL/6 mice were used for these experiments.

### Methods:

### 1. Dry Eye Model

Dry eye was induced by placing mice into a controlled environment chamber (CEC) as previously described (Barabino et al., 2005). Briefly, mice placed in CEC were exposed to relative humidity (RH)<25%, temperatures from 20° ° C. to 22° ° C., and airflow of 15 L/min for 12 hours per day. Non-stressed (NS) mice without stress-induced dry eye were maintained in a normal environment (RH>50%, no airflow, temperature of 21° ° C. to 23° C.) for the same duration.

### 2. Corneal Fluorescein Staining

Animals were anesthetized by intraperitoneal injection of a mixture of zoletil (6 mg/kg) and xylazine (3 mg/kg). Corneal epithelial damage was determined using topical fluorescein (Fluor-I-Strip, Averst Laboratories, Philadelphia, PA) staining. Corneal fluorescein staining was examined with a slit-lamp biomicroscope under cobalt blue light and photographed with a digital camera. Corneal dye staining is scored as follows: no spot staining, score 0); when less than one-third of the cornea is stained, score 1: when two-thirds or less of the cornea is stained, score 2: when more than two-thirds of the cornea is stained, score 3 (Horwath-Winter J 2013).

### 3. Measurement of Tear Production

Tear production was measured with phenol red-impregnated cotton thread (Zone-Quick: Oasis. Glendora, Canada). The validity of the assay was verified as previously described (Dursun et al., 2002). Hold the cotton thread with jeweler forceps and place it into the lateral canthus for 60 seconds. Tear production is expressed as the number of millimeters of cotton thread that turns red after being moistened by tears.

### 4. PAS Staining of Goblet Cells

After the animals were euthanized, the eyes were surgically excised, fixed in 10% formalin, embedded in paraffin, and cut into 5-μm sections. These sections were stained with periodic acid Schiff reagent (PAS: Sigma-Aldrich) to measure goblet cells in the superior and inferior conjunctiva and were examined and photographed using a microscope equipped with a digital camera. PAS-positive goblet cells in the conjunctiva were measured in five sections from each eye.

### Example 18.1—Restoration of Ocular Surface Damage Induced by Desiccation Stress by Topical Treatment with the Formulations of the Present Invention

To determine whether the formulation of the present invention has a therapeutic effect on desiccation stress (DS)-induced ocular surface defects, mice were housed in a controlled environment chamber (CEC) for 14 days to develop ocular surface ruptures. After 14 days in CEC, we used mice with fluorescence scores exceeding 2 for the first experiment. Subsequently, topical dry eye treatment was performed for an additional 5 days (3 times/day) with Formulations 1-15 of the invention or vehicle (1% CMC in BSS) while maintaining the same dry stress regimen.

On day 0, the mice had significantly lower mean tear volumes compared to unstressed (NS) mice, as measured by the cotton thread test. Tear productions in the eyes were significantly increased after 5 days of treatment of mice with formulations 1-15 of the invention, as compared to the vehicle group.

### Example 18.2—the Formulations of the Invention Partially Restore the Number of Goblet Cells in the Conjunctiva

Goblet cells are found primarily in the superficial epithelium of the conjunctival fornix and are responsible for the production of mucoid tears. Periodic acid Schiff (PAS) staining of NS eyes showed that goblet cells were in a continuous and uniform pattern in the conjunctival epithelium. However, after 14 days of desiccation stress (day 0), PAS staining of the conjunctiva showed that the number of goblet cells was significantly reduced compared with the NS group. After treatment with formulations 1 to 15 of the present invention or vehicle for 5 days, the numbers of conjunctival goblet cells in the eyes treated with the formulation of the present invention were significantly increased compared with the vehicle-treated control. In conclusion, treatments with the formulations of the invention did rescue (increase) the number of goblet cells.

### Example 18.3—Preventing ocular surface damage induced by desiccation stress by topical treatments with formulations of the present invention

To investigate whether the inventive formulations can inhibit DS-induced corneal epithelial damages, we subjected mice to a 14-day desiccation stress regimen and topically treated these mice with inventive formulations 1-15 three times a day. After 14 days, corneal epithelial defects were assessed using fluorescein dye staining. The results showed that the corneal fluorescein staining fractions were significantly elevated in vehicle-treated eyes, as compared with eyes treated with formulations of the invention. These results suggest that the formulations of the present invention also exhibit a preventive effect on the ocular surface against drying stress.

### Example 18.4—Formulations of the Present Invention Inhibit Inflammatory Responses Induced by Drying Stress

It has been suggested in experimental animals that inflammation increases ocular surface damages in dry eyes induced by desiccation stress (Luo et al., 2004; De Paiva et al., 2006). Among pro-inflammatory mediators, amelioration of desiccation stress-induced dry eye has been reported in mice pretreated with TNF-α or interleukin-1 (IL-1) blockers (Ji YW 2013; Okanobo A2012). After mice were housed in CEC for 14 days (set as day 0; untreated mice), mRNA levels of pro-inflammatory mediators, including IL-18, TNF-α, IL-6 and MCP-1 were significantly up-regulated by 2-4 times, respectively, as compared with mice housed in a non-stressed (NS) environment. However, as compared with the vehicle-treated group, mice topically treated with formulations 1-15 of the present invention for 5 days had the mRNA expressions of IL-IB. TNF-α, IL-6 and MCP-1 in the eyes increased by 1-2.5 times, respectively. In conclusion, these results indicate that the formulations of the present invention reduce the DS-induced ocular inflammatory responses.

Obviously, based on the above description of the present invention, one having common technical knowledge and common means in the field can devise various other modifications, replacements, or changes without departing from the above basic technical idea of the present invention. Those skilled in the art can understand that various features of the technical solutions of the present invention described herein can be appropriately combined as needed.

1. A composition comprising: a PEDF-derived short peptide (PDSP), a stabilizer, and a buffer, wherein the composition is in a liquid form, wherein a pH of the composition is in a range of 4-9, wherein the stabilizer is a copolymer of vinylpyrrolidone and vinyl acetate, wherein the buffer is a citric acid buffering system, a phosphate buffering system, a boric acid buffering system, a histidine buffering system, or a combination thereof.

2. The composition according to claim 1, wherein the composition further comprises an auxiliary reagent selected from an osmolarity regulator, a bacteriostatic agent, a suspension agent, or a combination thereof.

3. The composition according to claim 2, wherein the osmolarity regulator is selected from the group consisting of sorbitol, sodium chloride, potassium chloride, glucose, boric acid, and a combination thereof; wherein a concentration of the osmolarity regulator is 0.005%-5% w/v.

4. The composition according to claim 2, wherein the bacteriostatic agent is selected from the group consisting of a quaternary ammonium salt, an organic mercury, an amidine, an alcohol, an ester, and a combination thereof, wherein a concentration of the bacteriostatic agent is 0.004%-0.025% w/v.

5. The composition according to claim 1, wherein the pH of the composition is in a range of 6-8, wherein the composition is in a form of a solution or a suspension.

6. The composition according to claim 1, wherein the PDSP is selected from SEQ ID NO: 1, 2, 3, 5, 6, 8, 9, or a combination thereof, wherein a concentration of PDSP is 0.001%-5% w/v.

7. The composition according to claim 1, wherein a concentration of the stabilizer is 0.1%-4% w/v.

8. The composition according to claim 1, wherein a concentration of the buffer is 1 mM-200 mM.

9. A method for preparing the composition of claim 1, comprising:
(a) adding the stabilizer and the buffer;
(b) adjusting the pH to the range of 4-9; and
(c) adding the PEDF-derived short peptide (PDSP).

10. A method for the treatment and/or prevention of a disease, comprising administering to a subject in need thereof the composition according to claim 1, wherein the disease is selected from the group consisting of muscle damage, alopecia and/or hair depigmentation, osteoarthritis, skin aging, liver cirrhosis, eye disease, and a combination thereof.


SEQUENCE LISTING (9 sequences)

SEQ ID NO: 1 - protein, 39 aa, Artificial Sequence
  Synthetic
LSVATALSALSLGAEQRTESIIHRALYYDLISSPDIHGT

SEQ ID NO: 2 - protein, 34 aa, Artificial Sequence
  Synthetic
ALSALSLGAEQRTESIIHRALYYDLISSPDIHGT

SEQ ID NO: 3 - protein, 29 aa, Artificial Sequence
  Synthetic
SLGAEQRTESIIHRALYYDLISSPDIHGT

SEQ ID NO: 4 - protein, 25 aa, Artificial Sequence
  Synthetic
EQRTESIIHRALYYDLISSPDIHGT

SEQ ID NO: 5 - protein, 24 aa, Artificial Sequence
  Synthetic
SLGAEQRTESIIHRALYYDLISSP

SEQ ID NO: 6 - protein, 20 aa, Artificial Sequence
  Synthetic
SLGAEQRTESIIHRALYYDL

SEQ ID NO: 7 - protein, 18 aa, Artificial Sequence
  Synthetic
EQRTESIIHRALYYDLIS

SEQ ID NO: 8 - protein, 29 aa, Artificial Sequence
  Synthetic
SLGAEHRTESVIHRALYYDLITNPDIHST

SEQ ID NO: 9 - protein, 20 aa, Artificial Sequence
  Synthetic
SLGAEHRTESVIHRALYYDL